(19) United States
(12) Patent Application Publication
(10) Pub. No.: US 2026/0294870 A1
(43) Pub. Date: Oct. 1, 2026

(54) INDOLE LACTATE FOR THE TREATMENT OF MULTIPLE SCLEROSIS

(71) Applicant: The Johns Hopkins University, Baltimore, MD (US)

(72) Inventors: Pavan Bhargava, Baltimore, MD (US); Peter A. Calabresi, Baltimore, MD (US); Michael D. Kornberg, Baltimore, MD (US); Kathryn Fitzgerald, Baltimore, MD (US)

(21) Appl. No.: 19/477,847

(22) PCT Filed: May 1, 2024
(86) PCT No.: PCT/US2024/027239

§ 371 Date: Oct. 23, 2025

Related U.S. Application Data
(60) Provisional application No. 63/499,351, filed on May 1, 2023.

(51) Int. Cl. A61K 31/405; A61K 31/136; A61K 31/137; A61K 31/225; A61K 31/277; A61K 31/397; A61K 31/4015; A61K 31/7076; A61K 39/395; A61K 45/06; A61P 25/28

(52) U.S. Cl. CPC A61K 31/405; A61K 31/136; A61K 31/137; A61K 31/225; A61K 31/277; A61K 31/397; A61K 31/4015; A61K 31/7076; A61K 39/3955; A61K 45/06; A61P 25/28

(57) ABSTRACT

The use of indole lactate for treating Multiple Sclerosis is disclosed. Methods herein include a method for treating multiple sclerosis (MS) in a subject in need of treatment thereof, the methods enclosed herein comprise administering to the subject a therapeutically effective amount of indole lactate or indole-3-lactic acid. In certain aspects, administration of indole lactate or indole-3-lactic acid repairs demyelination of neurons and reduces inflammation in the subject with MS. In certain aspects, the administering comprises oral or intravenous administration. In certain aspects, the MS is selected from relapsing-remitting MS, secondary progressive MS, primary-progressive MS, and progressive-relapsing MS. In particular aspects, the MS is relapsing-remitting MS or secondary¬progressive MS.

### FIELD

The invention relates to methods for treating multiple sclerosis.

### BACKGROUND

Multiple sclerosis (MS) is an autoimmune disease of the central nervous system with inflammatory and neurodegenerative components. Reich et al., 2018. There are approximately one million patients with MS in the U.S. alone and given the relatively early onset of this disease compared to other chronic neurological conditions, this results in an enormous cost to society. Wallin et al., 2019. The majority of patients (85%) have a relapsing-remitting course at onset, however 50% of them will transition to a progressive phase of the disease over about two decades. While there are a multitude of treatment options for the relapsing-remitting phase of the disease, treatments for progressive MS are limited in number and efficacy. Even for the relapsing phase of the disease, the majority of treatments are associated with significant adverse effects and do not address processes that are abnormal in the disease. Thus, there is a major need for treatments that are low-risk, well-tolerated and address the underlying pathological processes at play in MS.

Besides genetic factors that predispose to development of MS, several environmental factors are known to play a role in the development of the disease. Munger and Ascherio, 2011; Beecham et al., 2013. The gut microbiota in people with MS demonstrates alterations compared to healthy individuals. Jangi et al., 2016; Wang and Kasper, 2013. Indeed, some of the changes have been linked to the role of these specific bacteria in altering the immune system, while the precise mechanism by which others might play a role in MS is unknown. Besides the direct effects on immune cells in the gut, bacteria can affect a variety of cells in the body through metabolites produced by them being absorbed into human circulation. Marcobal et al., 2013; Bhargav et al., 2019.

Several groups have shown that there are alterations in the levels of several circulating metabolites in people with MS when compared to healthy individuals. Fitzgerald et al., 2021; Levi et al., 2021. Some of the most important and consistent changes identified are among metabolites that are produced primarily by the gut microbiota. For example, it has been found that gut microbiota derived metabolites of aromatic amino acids, such as tryptophan, were associated with disease severity in people with MS and had direct effects on peripheral myeloid cells. Fitzgerald et al., 2021.

### SUMMARY

The presently disclosed subject matter provides a method for treating multiple sclerosis (MS) in a subject in need of treatment thereof, the method comprising administering to the subject a therapeutically effective amount of indole lactate or indole-3-lactic acid.

In certain aspects, administration of indole lactate or indole-3-lactic acid repairs demyelination of neurons and reduces inflammation in the subject with MS. In certain aspects, the administering comprises oral or intravenous administration.

In certain aspects, the MS is selected from relapsing-remitting MS, secondary-progressive MS, primary-progressive MS, and progressive-relapsing MS. In particular aspects, the MS is relapsing-remitting MS or secondary-progressive MS.

In certain aspects, the method comprises reducing a severity of the MS or reducing one or more symptoms of MS in the subject. In particular aspects, the one or more symptoms of MS are selected from fatigue, vision problems, including optic neuritis, numbness and tingling, muscle spasms, stiffness and weakness, mobility problems, pain, including neuropathic pain, cognitive dysfunction, depression and anxiety, sexual problems, bladder and bowel problems, speech difficulties (dysphagia), and swallowing difficulties (dysarthria).

In certain aspects, administration of indole lactate or indole-3-lactic acid reduces one or more of T cell proliferation, T cell activation, pro-inflammatory cytokine production, including IFN-g and IL-17 macrophage infiltration, and nitric oxide from macrophages. In certain aspects, administration of indole lactate or indole-3-lactic acid promotes differentiation of oligodendrocyte precursor cells (OPCs) to myelin-producing oligodendrocytes. In certain aspects, it is thought that administration of indole lactate or indole-3-lactic acid repairs demyelination in a subject afflicted with MS.

In certain aspects, the method further comprises administering indole lactate or indole-3-lactic acid in combination with one or more additional treatments for MS. In particular aspects, the one or more additional treatments for MS are selected from a corticosteroid, plasma exchange (plasmapheresis), beta interferon drugs, glatiramer acetate, natalizumab, ocrelizumab, alemtuzumab, mitoxantrone, fingolimod, dimethyl fumarate, teriflunomide, cladribine, diroximel fumarate, and 3iponimod (Mayzent).

Certain aspects of the presently disclosed subject matter having been stated herein above, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 demonstrates that ILA levels and ILA/IAA ratio are correlated with disability measures—Walking speed, MDT (manual dexterity test), processing speed test and patient reported outcomes—PDDS (patient determined disease steps) and NeuroQOL (neurological quality of life) measures. Partial correlation coefficients adjusted for age, sex and disease subtype are shown;

FIG. 2 demonstrates that levels of ILA at baseline predict future development of T2 and gadolinium enhancing lesions in the brain and spinal cord during follow up;

FIG. 3 demonstrates that indole lactate reduces the severity of neuroinflammation in experimental autoimmune encephalomyelitis. Disease severity was reduced significantly in both low-dose and high-dose indole lactate groups compared to vehicle. (****-p<0.0001);

FIG. 4 demonstrates that oral indole lactate reduces the severity of neuroinflammation in experimental autoimmune encephalomyelitis. Disease severity was reduced significantly by oral indole lactate compared to vehicle. (****-p<0.0001). We also saw reduction in immune cell infiltration on IHC and flow cytometry analysis;

FIG. 5 demonstrates that indole lactate reduces T cell proliferation, activation and pro-inflammatory cytokine production. Murine MOG specific T cells were isolated and stimulated with antigen+/1 Th17 polarizing conditions for 72 hours followed by flow-cytometry to analyze viability, proliferation and Th17 polarization in presence of vehicle or varying doses of ILA;

FIG. 6 demonstrates that indole lactate promotes differentiation of murine oligodendrocyte precursor cells. We noted increased differentiation of OPCs with ILA treatment compared to vehicle. Also, as expected, PDGF-a withdrawal resulted in significant OPC differentiation, which was further potentiated by either ILA or T3. (* p<0.05, ** p<0.01, *** p<0.005). Similar results also were obtained when examining qPCR for MBP gene;

FIG. 7 demonstrates that indole lactate promotes differentiation of human embryonic stem cell derived oligodendrocyte precursor cells. Indole lactate treatment at 300 μM and 100 μM promoted oligodendrocyte precursor cell differentiation based on production of luciferase which is a marker of MBP expression (a protein produced by myelinating oligodendrocytes). (* p<0.05, ** p<0.01);

FIG. 8A, FIG. 8B, and FIG. 8C demonstrate that ILA promotes remyelination in mice treated with cuprizone. FIG. 8A. Schematic representation of Cuprizone experiments. FIG. 8B. Representative Black-Gold staining images through the corpus callosum. FIG. 8C. Quantification of myelination within the corpus callosum based on Black-Gold staining. * p<0.05, ***p<0.005;

FIG. 9 demonstrates that oral ILA promotes remyelination in mice treated with cuprizone. Schematic representation of Cuprizone experiments with oral ILA administration is shown on left. Representative Black-Gold and Fluoromyelin staining images through the corpus callosum are shown in the middle along with quantification of myelination within the corpus callosum based on staining. On the right are electron micrographs of the corpus callosum that show higher proportion of remyelinated axons in the ILA group compared to vehicle. * p<0.05, ***p<0.005;

FIG. 10A and FIG. 10B demonstrate that AhR in human and primary murine OPCs. FIG. 10A. Schematic of treatment paradigm of human ES-derived OPCs with ILA or vehicle. FIG. 10B. CYP1B1 expression increased in OPCs treated with high-dose ILA. C. ICC of neonatal murine OPC cultures demonstrates presence of AhR (green) in Olig2+ (oligodendrocyte lineage marker) cells and in Olig2+ PDGFRα+ (OPCs) cells.

FIG. 11 demonstrates ILA likely mediates effects on OPCs through AhR. FIG. 11A. is a comparison of OPC differentiation in WT and AhR KO mice that shows loss of pro-differentiation effect in AhR KO OPCs. FIG. 11B shows in human ES-derived OPCs at low dose of ILA an AhR inhibitor reverses the effects of ILA on OPC differentiation. * p<0.05, ** p<0.01;

FIG. 12A, FIG. 12B, FIG. 12C, FIG. 12D, FIG. 12E, FIG. 12F, FIG. 12G, FIG. 12H, and FIG. 12I show: FIG. 12A. Schematic of product of ILA and IAA and the change in levels with ILA supplementation in naïve mice. FIG. 12B. Clinical scores and plasma ILA and IAA levels at end of experiment in mice on vehicle or oral ILA supplementation. FIG. 12C. Change in ILA/IAA ratio. FIG. 12D. Correlation of ILA/IAA ratio and cumulative EAE score. FIG. 12E. Gut microbiota composition in vehicle and ILA groups at various time points (n=10 each group). FIG. 12F. Change in alpha diversity over the course of EAE.

FIG. 12G. Change in specific bacterial abundance over the course of experiment in both groups. FIG. 12H. Change in functional pathways with ILA supplementation. FIG. 12I. Correlation of IAA levels with acetylene degradation pathway enrichment; and

FIG. 13A, FIG. 13B, FIG. 13C, and FIG. 13D demonstrate that indole acetate prevents remyelination in the cuprizone model of demyelination. FIG. 13A. Experimental paradigm. FIG. 13B. quantification of area of myelination in the corpus callosum. FIG. 13C. Staining for mature oligodendrocytes (Olig2+CC-1+ and OPCs (PDGFRα+). FIG. 13D. Staining for astrocytes (GFAP), immune cells (CD45) and microglia (Iba1).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Multiple sclerosis (MS) is the most common disabling neurological disease of young adults with symptom onset generally occurring between the ages of 20 to 40 years. In MS, the immune system attacks myelin, which makes up a protective sheath that coats axons, in the central nervous system (brain, optic nerves, and spinal cord).

There are four main types of MS, including relapsing-remitting MS, secondary-progressive MS, primary-progressive MS, and progressive-relapsing MS. Most people with MS are initially diagnosed with relapsing-remitting MS, in which symptoms arise in the form of an attack, a relapse, or exacerbation, followed by remission. Individuals with relapsing-remitting MS may progress to secondary progressive MS during which they develop gradual, steady symptoms and deterioration in function over time. Primary-progressive MS and progressive-relapsing MS are less common.

MS can cause a wide range of symptoms and affect any part of the body, with each person afflicted with MS being affected differently. The symptoms of MS are unpredictable. Some people's symptoms develop and worsen steadily over time, while for others they come and go. Periods when symptoms worsen are known as relapses, whereas periods when symptoms improve or disappear are known as remissions.

Some of the most common symptoms of MS include, but are not limited to: fatigue, vision problems, including optic neuritis, numbness and tingling, muscle spasms, stiffness and weakness, mobility problems, pain, including neuropathic pain, cognitive dysfunction, depression and anxiety, sexual problems, bladder and bowel problems, and speech (dysphagia) and swallowing difficulties (dysarthria).

There is no cure for MS, but treatments can reduce the number and severity of relapses and delay the long-term disability progression of the disease, including corticosteroids and plasma exchange (plasmapheresis). Disease-modifying treatments include beta interferon drugs, glatiramer acetate, natalizumab, ocrelizumab, alemtuzumab, mitoxantrone, fingolimod, dimethyl fumarate, teriflunomide, cladribine, diroximel fumarate, and 7iponimod (Mayzent). Despite these current methods of treating MS, there is a need for improved treatments for MS that are low-risk, well-tolerated, and address the underlying pathological processes at play in MS.

Accordingly, in some embodiments, the presently disclosed subject matter provides a method for treating multiple sclerosis (MS) in a subject in need of treatment thereof, the method comprising administering to the subject a therapeutically effective amount of indole lactate or indole-3-lactic acid.

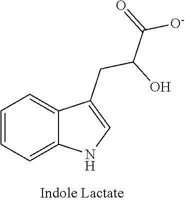 [SMILES: O=C([O-])C(O)Cc1c[nH]c2ccccc12]

Indole lactate, indole-3-lactic acid, and ILA are used interchangeably herein and throughout. Under physiological conditions, the compound is in the form of lactate. Indole lactate can be administered as indole lactate or indole-3-lactic acid. In certain embodiments, administration of indole lactate or indole-3-lactic acid repairs demyelination of neurons and reduces inflammation in the subject with MS. In certain embodiments, the administering comprises oral or intravenous administration.

In certain embodiments, the MS is selected from relapsing-remitting MS, secondary-progressive MS, primary-progressive MS, and progressive-relapsing MS. In particular embodiments, the MS is relapsing-remitting MS or secondary-progressive MS.

In certain embodiments, the method comprises reducing a severity of the MS or reducing one or more symptoms of MS in the subject. In particular embodiments, the one or more symptoms of MS are selected from fatigue, vision problems, including optic neuritis, numbness and tingling, muscle spasms, stiffness and weakness, mobility problems, pain, including neuropathic pain, cognitive dysfunction, depression and anxiety, sexual problems, bladder and bowel problems, speech difficulties (dysphagia), and swallowing difficulties (dysarthria).

In certain embodiments, administration of indole lactate or indole-3-lactic acid reduces one or more of T cell proliferation, T cell activation, pro-inflammatory cytokine production including IFN-g and IL-17, macrophage infiltration, and nitric oxide from macrophages. In certain embodiments, administration of indole lactate or indole-3-lactic acid promotes differentiation of oligodendrocyte precursor cells (OPCs) to myelin-producing oligodendrocytes. In certain embodiments, without wishing to be bound to any one particular theory, it is thought that administration of indole lactate or indole-3-lactic acid repairs demyelination in a human subject afflicted with MS. This hypothesis is based on our ability to repair demyelination in animal models of MS.

In certain embodiments, the method further comprises administering indole lactate or indole-3-lactic acid in combination with one or more additional treatments for MS. In particular embodiments, the one or more additional treatments for MS are selected from a corticosteroid, plasma exchange (plasmapheresis), beta interferon drugs, glatiramer acetate, natalizumab, ocrelizumab, alemtuzumab, mitoxantrone, fingolimod, dimethyl fumarate, teriflunomide, cladribine, diroximel fumarate, and siponimod (Mayzent).

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The “therapeutically effective amount” of an active agent refers to the amount necessary to elicit the desired biological response. As will be appreciated by those of ordinary skill in this art, the effective amount of an agent may vary depending on such factors as the desired biological endpoint, the agent to be delivered, the makeup of the pharmaceutical composition, the target tissue, and the like.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Example 1

### Indole Lactate for the Treatment of Multiple Sclerosis

### 1.1 Overview

In previous studies, we have identified that people afflicted with multiple sclerosis (MS) have lower levels of indole lactate, which is a metabolite produced by gut microbiota from metabolism of tryptophan. The levels of this metabolite also are associated with disease severity in people afflicted with MS.

We found that indole lactate supplementation reduced disease severity in an animal model of MS that has been used to identify several currently used treatments for MS. We also noted that indole lactate reduced pro-inflammatory cytokine production from T cells in culture and the production of nitric oxide in macrophages. Without wishing to be bound to any one particular theory, these effects, which are anti-inflammatory, might be one explanation for the effect noted in the animal model.

Besides the anti-inflammatory effects, we also tested the effects on oligodendrocyte precursor cells in vitro. These cells are present in the brain and can differentiate into oligodendrocytes and help repair demyelination, which is the hallmark of MS. We found using both human and murine oligodendrocyte precursor cells that indole lactate promoted differentiation of these cells to myelin-producing oligodendrocytes. This mechanism also would be beneficial in the setting of MS since it could promote repair of existing damage.

### 1.2 Scope

The Example further evaluates the mechanism of action of this metabolite by which it mediates the effects noted hereinabove. Given that this metabolite is found commonly in fermented foods, we expect that it will be safe in humans.

### 1.3 Results

1.3.1 Indole Lactate Levels are Decreased in People with MS

Circulating levels of indole lactate were measured using HPLC and tandem mass spectrometry in a large cohort of people with MS and healthy controls. We noted significant reduction in the levels of indole lactate in the blood of people with MS compared to controls with a mean difference of −5.76 (95% CI: −7.19 to −4.33, p=2.61E−15). Similar results also have been reported in studies from other centers. Levi et al., 2022.

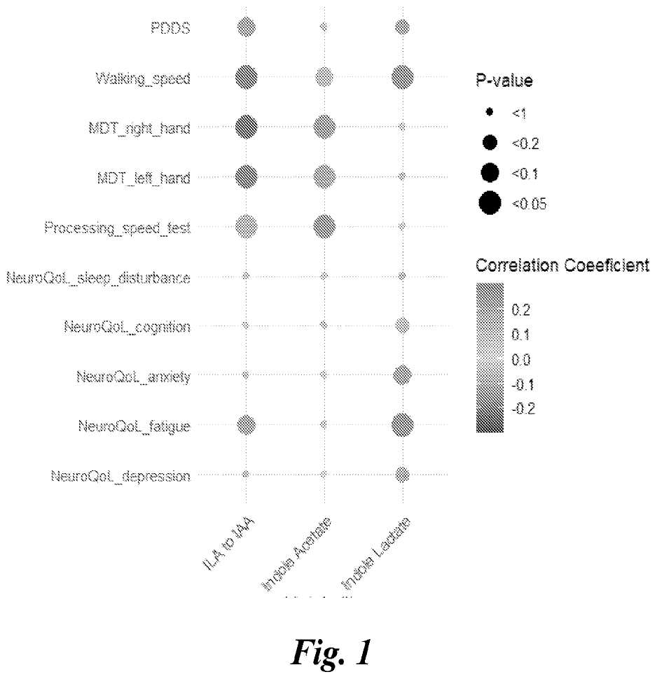

Indole lactate levels and the ratio of indole lactate to indole acetate (a metabolite in the alternative route of tyrptophan metabolism in the gut), in a subset of our cohort (n=280 people with MS) with various clinical and patient reported outcomes were associated with various measures of disability and patient reported outcomes (FIG. 1).

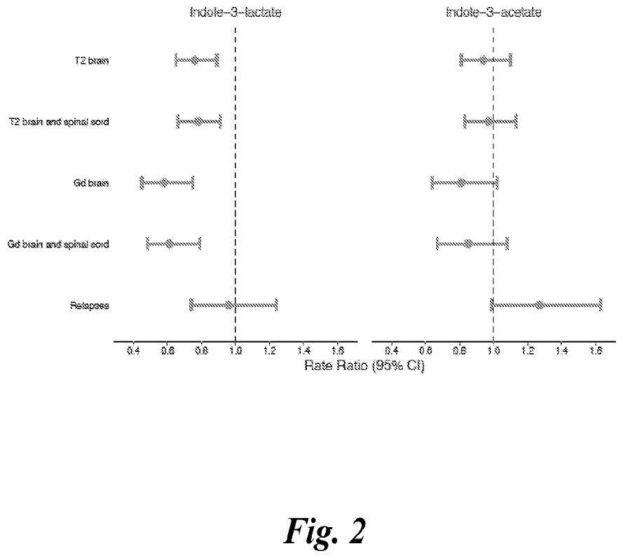

In an independent cohort of 380 patients with pediatric onset MS with a median follow up of approximately 4 years, the levels of indole lactate at baseline were associated with a lower subsequent risk of new T2 or gadolinium-enhancing MRI lesions in both the brain and the spinal cord (FIG. 2).

### 1.3.2 Indole Lactate Reduces Severity of Neuroinflammation in an Animal Model of MS

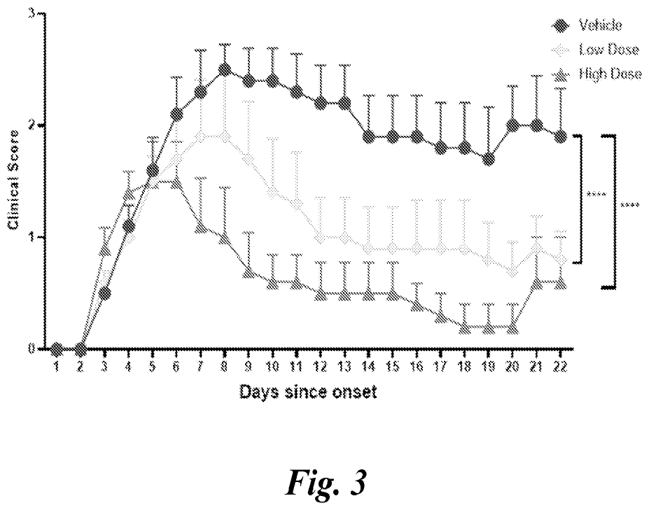

Mice (C57/BL6, 8-9 week old) were immunized with MOG35-55 peptide and CFA and received IP injection of PTX on day 0 and 2. When mice demonstrated signs of illness they were randomized to either vehicle, low-dose (10 mg/kg) or high-dose (20 mg/kg) indole lactate IP twice daily. Mice were weighed and scored daily using the following scale (0-normal, 1-limp tail, 2-hind-limb weakness, 3-hind-limb paralysis, 4-fore-limb weakness, 5-death). We noted a significant dose-dependent reduction in disease severity with indole lactate compared to vehicle treatment (FIG. 3).

We subsequently performed additional experiments in which we induced EAE using the same paradigm, however at day 7 post-immunization mice were randomized to receive ILA orally in drinking water (ILA 2 mM) or acidified water (adjusted to a similar pH) for the next 3 weeks. At four weeks post-immunization mice were euthanized and tissues were collected and processed for immunohistochemistry. We stained sections of the spinal cord to assess for extent of demyelination and for infiltration of immune cells.

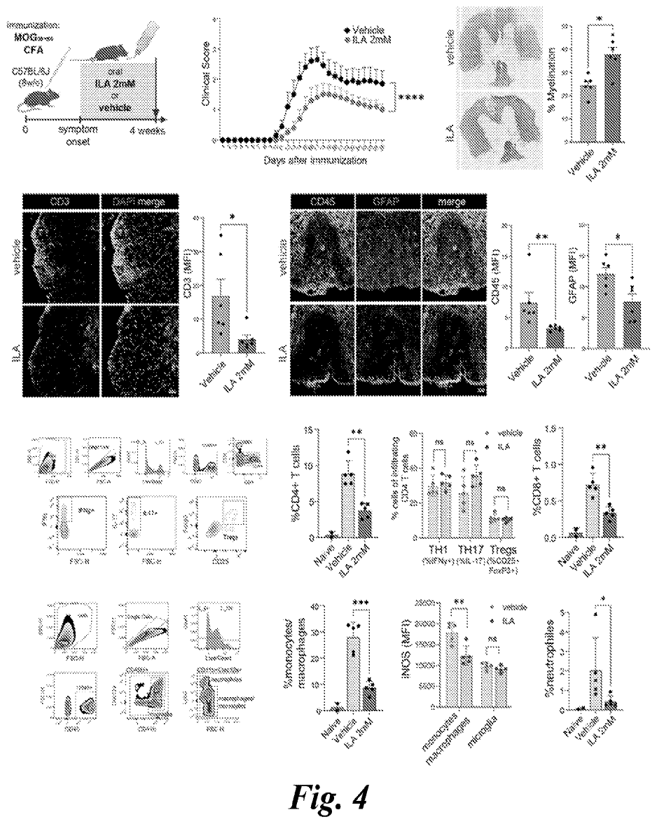

In these experiments we noted reduced severity of EAE with ILA administered orally. We also noted reduced extent of demyelination and immune cell infiltration on IHC in the ILA group. Additionally, we repeated experiments and euthanized mice at the peak of disease to obtain spinal cord tissues for flow cytometric analyses. We noted reduced infiltration of T cells and macrophages with a shift towards less iNOS producing inflammatory macrophages. These results are shown in FIG. 4.

### 1.3.3 Indole Lactate Decreases T Cell Proliferation and Cytokine Production

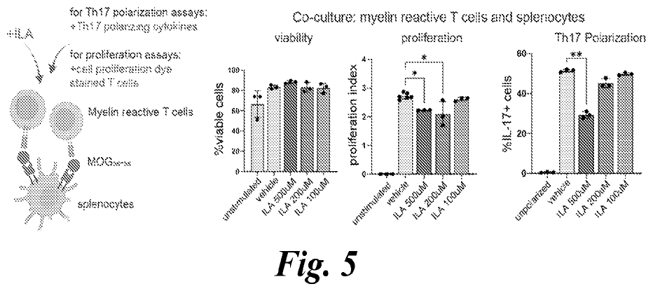

T cells were isolated from spleen and lymph nodes of 2D2 TCR transgenic C57/BL6 mice using negative selection (STEMCELL CD4 T cell isolation kit). T cells were then cultured in c-RPMI with MOG35-55 peptide in the presence of irradiated antigen presenting cells with or without additional cytokines to polarize the cells to a Th17 phenotype for 72 hours and then underwent flow-cytometry to analyze proliferation and Th17 polarization. We noted that indole lactate reduced T cell proliferation and Th17 polarization in a dose-dependent manner (FIG. 5).

### 1.3.4 Indole Lactate Promotes Differentiation of Oligodendrocyte Precursor Cells

### 1.3.4.1 Effect of Indole Lactate on Murine Oligodendrocyte Precursor Cells

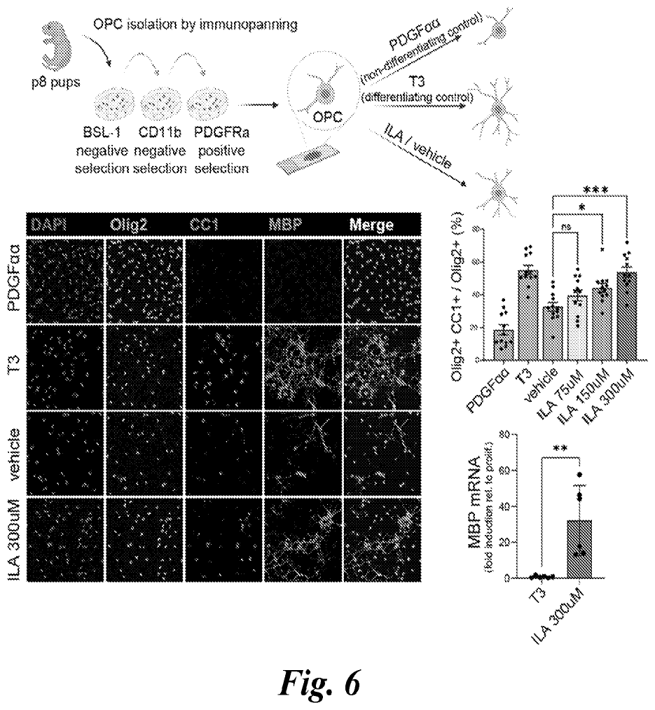

We tested the effect of several doses of indole lactate on differentiation of oligodendrocyte precursor cells isolated using immune-panning from neonatal mice. Compared to control conditions or OPCs treated with PDGF-a, we noted a dose-dependent increase in OPC differentiation as assessed by the expression of a marker for mature oligodendrocytes (CC1) using immunocytochemistry. As noted in FIG. 6, there were significantly higher proportion of Olig2 and CC1 double-positive cells in the ILA 150-μM and 300-μM conditions compared to vehicle and the proportion of these cells in these condition was similar to that seen using T3 (hormone known to promote OPC differentiation). Similar results also were noted in independent experiments where RNA was isolated from OPC cultures and qPCR performed for a marker of OPC differentiation-myelin basic protein. We noted significantly higher expression of MBP with ILA 300 μM with low dose T3 compared to T3 alone.

### 1.3.4.2 Effect of Indole Lactate on Human Embryonic Stem Cell Derived Oligodendrocyte Precursor Cells

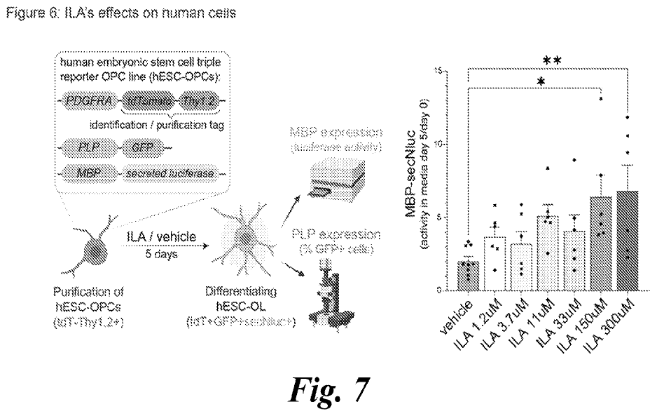

We also tested the effect of indole lactate on differentiation of human embryonic stem cell derived oligodendrocyte precursor cells. In this system, the cells produce luciferase under control of the promoter for MBP. Thus, OPC differentiation can be measured using the quantity of luciferase produced by cells. We noted a significant increase in OPC differentiation as measured by Luciferase release in the ILA treatment conditions in a dose-dependent manner, as seen in FIG. 7.

### 1.3.5 Indole Lactate Promotes Remyelination In Vivo in a Toxic Model of Demyelination

We tested the ability of indole lactate to promote remyelination in the Cuprizone-induced model of demyelination. Mice were fed with 0.2% Cuprizone for 4 weeks to induce demyelination and then switched to regular chow for one week. At three weeks of Cuprizone feeding mice were randomized to treatment with ILA 20 mg/kg, ILA 40 mg/kg or vehicle for two weeks. At the end of this period, we obtained brain tissues and used Black Gold staining to stain myelin and evaluated myelination in the corpus callosum and noted significant increase in area of myelination in both ILA groups (FIG. 7) suggesting that ILA promotes remyelination in vivo, as well.

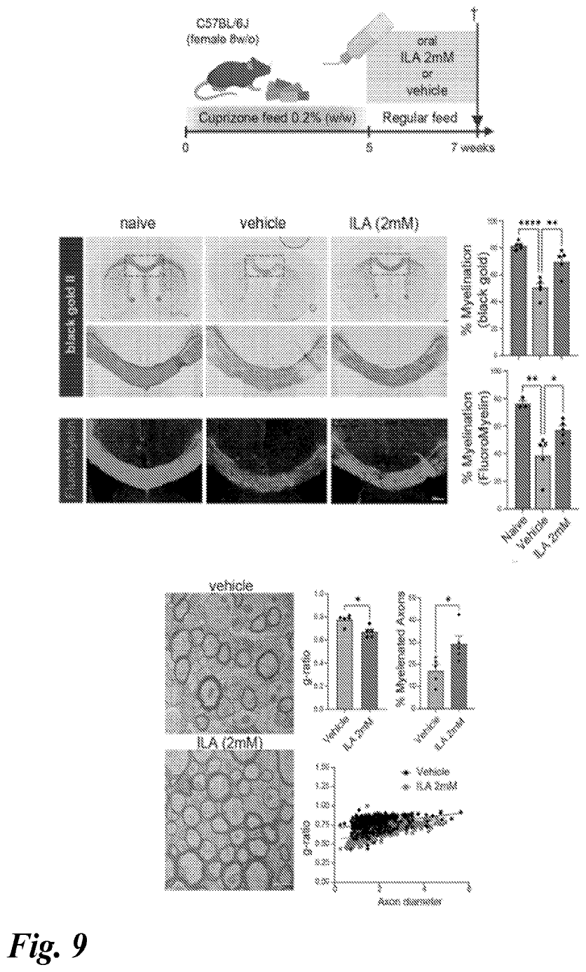

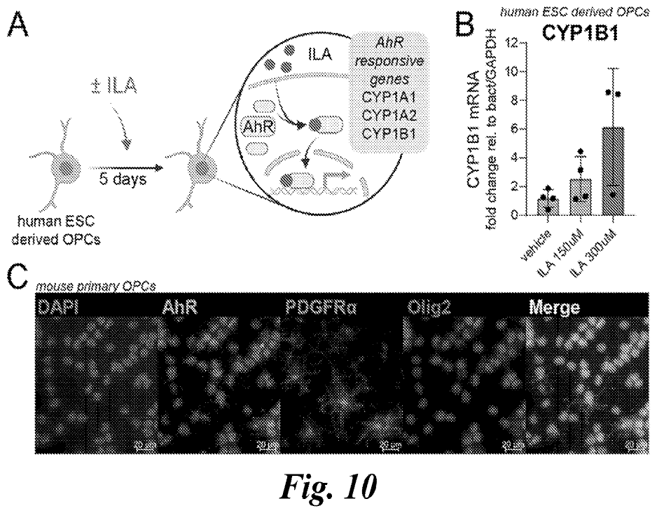

In subsequent experiments, we fed mice with 0.2% cuprizone for 5 weeks and then randomized them to ILA 2 mM in drinking water or acidified water as a control for 2 weeks. At the end of this period, we obtained brain tissues and evaluated extent of myelination in the corpus callosum and noted significantly increased remyelination in the ILA group compared to control (FIG. 9). We were able to demonstrate this with staining for myelin-black gold and fluoromyelin in addition to scanning EM imaging of the corpus callosum that showed significantly lower g ratio and higher proportion of myelinated axons in the ILA group compared to vehicle. These observations provide evidence that oral administration of ILA promotes remyelination in vivo in a mouse model of demyelination.

Indeed, the doses administered to the mice would translate to a human equivalent dose of approximately 400 mg daily.

### 1.3.6 Indole Lactate May Promote OPC Differentiation Through Effects at the Arylhydrocarbon Receptor (AhR)

We performed immunocytochemistry of murine OPCs to evaluate for the presence of AhR since indole metabolites have previously been shown to act at this receptor. We noted presence of AhR in Olig2+ and PDGFRα+ cells confirming that AhR is present in oligodendrocyte precursor cells. We also obtained RNA from human ES derived OPCs that had been treated with either vehicle or two different doses of ILA for 5 days. Using quantitative PCR we noted that there was increased expression of CYP1B1 (an AhR responsive gene) in the ILA groups compared to vehicle with a dose-response relationship suggesting that ILA activates the AhR and this may be responsible for the promotion of OPC differentiation with this metabolite.

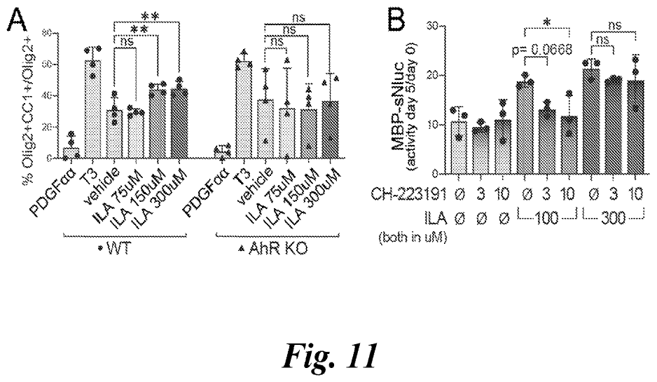

We further evaluated the mechanism of ILA using OPCs from mice that lack the Ahr gene—constitutive Ahr-KO mice. We noted that while ILA promoted OPC differentiation as expected in wild type (WT) mice, the effect was lost in Ahr KO mice suggesting that the effects of ILA on OPC differentiation are mediated through signaling at AhR (FIG. 11A).

We also tested the effects of blocking AhR signaling using a small molecules AhR antagonist. Here we saw that at low doses of ILA, the AhR antagonist blocked the pro-differentiating effects of ILA on human ES derived OPCs, though this effect was lost at high doses. (FIG. 11B) This observation suggests that signaling through AhR is at least partially responsible for the effects of ILA on human OPCs.

### 1.3.7 ILA Supplementation in Experimental Autoimmune Encephalomyelitis Alters the Gut Microbiota and Reduces the Production of Indole Acetate.

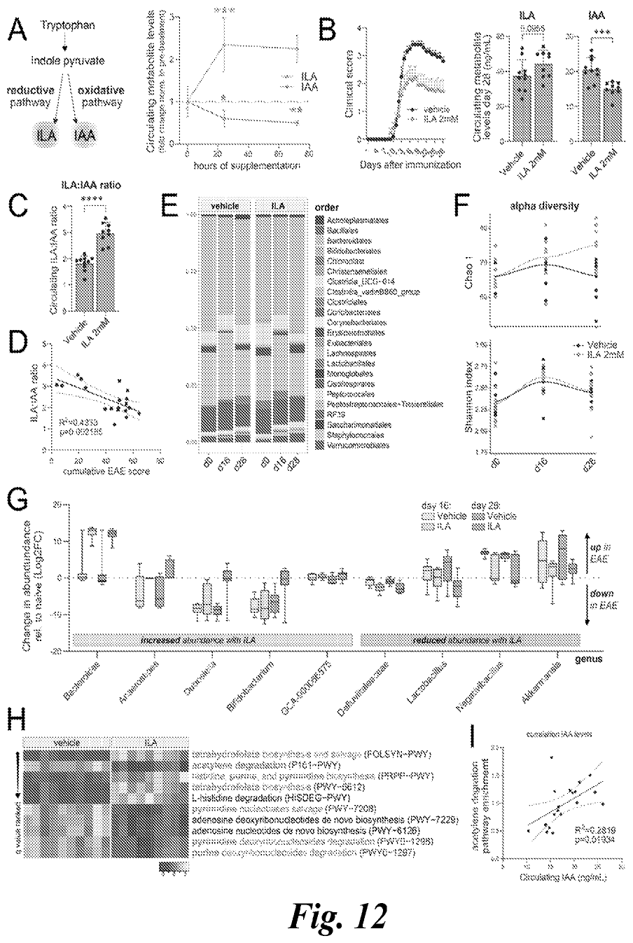

We examined the change in the gut microbiota using 16s rRNA sequencing at baseline, peak of disease and chronic disease in mice receiving ILA or vehicle orally. We noted a change in the overall composition of the gut microbiota in mice receiving ILA and a differential change in multiple bacteria compared to mice treated with vehicle (FIG. 12E-FIG. 12G).

As expected oral ILA supplementation increased circulating ILA levels in naïve mice (FIG. 12A). In EAE mice we noted an increase in ILA/IAA ratio mainly driven by reduction of IAA levels (FIG. 12B-FIG. 12C). Evaluating the functional changes in the gut microbiota with ILA supplementation we noted that ILA reduced acetylene degradation pathway in mice (FIG. 12H) and this would lead to less conversion of indole acetaldehyde to IAA. This pathway abundance correlated with IAA levels (FIG. 12I).

### 1.3.7 IAA Prevent Remyelination in a Toxic Model of Demyelination

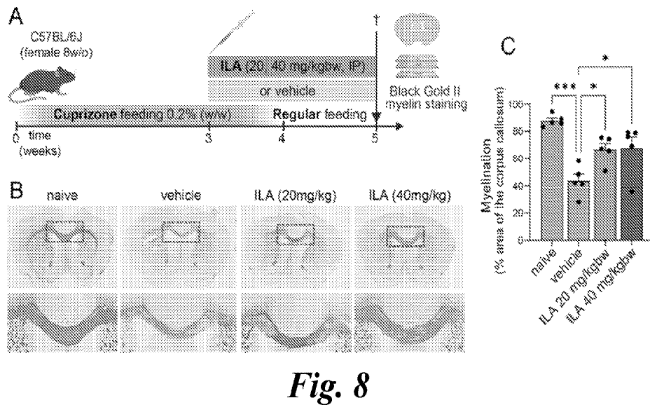

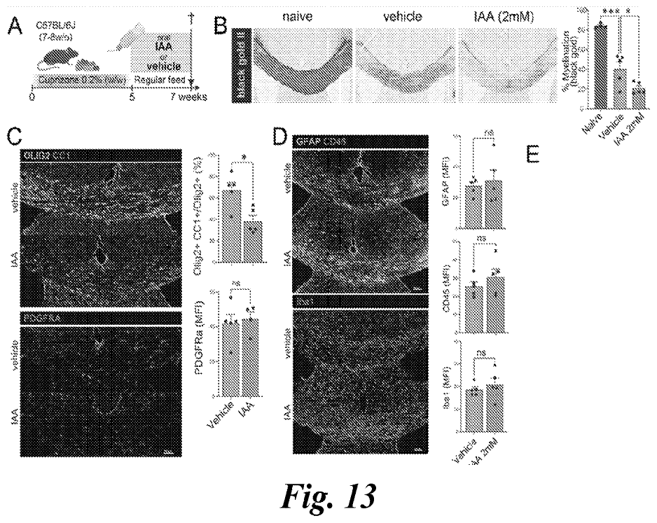

Since indole acetate is produced by an alternative pathway of gut microbiota metabolism and its levels were reduced by ILA production we tested its effects on remyelination in the cuprizone model of toxic demyelination as shown in FIG. 8 and FIG. 9 above. We noted that IAA supplementation led to reduced remyelination in mice (FIG. 13). This suggests that IAA is a toxic molecule and ILA supplementation not only has direct effects on the CNS but also helps indirectly by reducing levels of toxic molecules like IAA.

### 1.4 Summary

Based on these data, we have identified anti-inflammatory and reparative properties of indole lactate. Since this metabolite is decreased in circulation of people with MS, we propose that oral supplementation of indole lactate or indole-3-lactic acid will help to decrease disease activity and promote recovery in people with MS. Given that this metabolite is found in significant quantities in fermented foods, we do not anticipate that there will be significant safety concerns.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Reich D S, Lucchinetti C F, Calabresi P A. Multiple Sclerosis. Longo D L, editor. New England Journal of Medicine. 2018; 378:169-180.
Wallin M T, Culpepper W J, Campbell J D, et al. The prevalence of MS in the United States: A population-based estimate using health claims data. Neurology. 2019; 92:e1029-e1040.
Munger K L, Ascherio A. Prevention and treatment of MS: studying the effects of vitamin D. Multiple sclerosis (Houndmills, Basingstoke, England). 2011; 17:1405-1411.
Beecham A H, Patsopoulos N A, Xifara D K, et al. Analysis of immune-related loci identifies 48 new susceptibility variants for multiple sclerosis. Nature genetics. 2013; 45:1353-1360.
Jangi S, Gandhi R, Cox L M, et al. Alterations of the human gut microbiome in multiple sclerosis. Nature communications. 2016; 7:12015.
Wang Y, Kasper L H. The role of microbiome in central nervous system disorders. Brain, behavior, and immunity. Epub 2013 Dec. 25.
Marcobal A, Kashyap P C, Nelson T A, et al. A metabolomic view of how the human gut microbiota impacts the host metabolome using humanized and gnotobiotic mice. The ISME journal. Nature Publishing Group; 2013; 7:1933-1943
Bhargava P, Mische L, Smith M D, et al. Bile acid metabolism is altered in multiple sclerosis and supplementation ameliorates neuroinflammation. bioRxiv. Cold Spring Harbor Laboratory; Epub 2019 May 7: 627356.
Fitzgerald K C, Smith M D, Kim S, et al. Multi-omic evaluation of metabolic alterations in multiple sclerosis identifies shifts in aromatic amino acid metabolism. Cell Rep Med [online serial]. Cell Rep Med; 2021; 2. Accessed at: https://pubmed.ncbi.nlm.nih.gov/34755135/. Accessed Nov. 10, 2021.
Levi I, Gurevich M, Perlman G, et al. Potential role of indole lactate and butyrate in multiple sclerosis revealed by integrated microbiome-metabolome analysis. Cell Rep Med [online serial]. 2021; 2:100246. Accessed at: https://doi.org/10.1016/j.xcrm.2021.100246. Accessed Apr. 20, 2022.
Fitzgerald K C, Smith M D, Sotirchos E S, et al. Multiomic Evaluation of Metabolic Alterations in Multiple Sclerosis Identifies Shifts in Aromatic Amino Acid Metabolism. medRxiv. Cold Spring Harbor Laboratory Press; Epub 2021 Jan. 5: 2020.12.30.20249031.

Although the foregoing subject matter has been described in some detail by way of illustration and example for purposes of clarity of understanding, it will be understood by those skilled in the art that certain changes and modifications can be practiced within the scope of the appended claims.

1. A method for treating multiple sclerosis (MS) in a subject in need of treatment thereof, the method comprising administering to the subject a therapeutically effective amount of indole lactate.

2. The method of claim 1, wherein the MS is selected from relapsing-remitting MS, secondary-progressive MS, primary-progressive MS, and progressive-relapsing MS.

3. The method of claim 2, wherein the MS is selected from relapsing-remitting MS, primary-progressive MS, and secondary-progressive MS.

4. The method of claim 1, comprising reducing a severity of the MS or reducing one or more symptoms of MS in the subject.

5. The method of claim 4, wherein the one or more symptoms of MS are selected from fatigue, vision problems, including optic neuritis, numbness and tingling, muscle spasms, stiffness and weakness, mobility problems, pain, including neuropathic pain, cognitive dysfunction, depression and anxiety, sexual problems, bladder and bowel problems, and speech (dysphagia) and swallowing difficulties (dysarthria).

6. The method of claim 1, wherein administration of indole lactate reduces one or more of T cell proliferation, T cell activation, and pro-inflammatory cytokine production, including IFN-g and IL-17.

7. The method of claim 1, wherein administration of indole lactate promotes differentiation of oligodendrocyte precursor cells (OPCs).

8. The method of claim 1, wherein administration of indole lactate repairs demyelination in a subject afflicted with MS.

9. The method of claim 1, further comprising administering indole lactate in combination with one or more additional treatments for MS.

10. The method of claim 9, wherein the one or more additional treatments for MS are selected from a corticosteroid, plasma exchange (plasmapheresis), beta interferon drugs, glatiramer acetate, natalizumab, ocrelizumab, alemtuzumab, mitoxantrone, fingolimod, dimethyl fumarate, teriflunomide, cladribine, diroximel fumarate, and siponimod (Mayzent).
